(19) United States
(12) Patent Application Publication
(10) Pub. No.: US 2025/0072920 A1
(43) Pub. Date: Mar. 6, 2025

(54) VIDEO ASSISTED MEDICAL EXTRACTION DEVICE

(71) Applicant: Ali Osman, Beaumont, TX (US)

(72) Inventor: Ali Osman, Beaumont, TX (US)

(21) Appl. No.: 18/950,988

(22) Filed: Nov. 18, 2024

Related U.S. Application Data
(62) Division of application No. 17/469,621, filed on Sep. 8, 2021.
(60) Provisional application No. 63/075,345, filed on Sep. 8, 2020.

(51) Int. Cl. A61B 17/22; A61B 1/015; A61B 1/05; A61B 1/06; A61B 17/00; A61M 25/10; H04N 23/50; H04N 23/56; H04N 23/57

(52) U.S. Cl. CPC A61B 17/22032; A61B 1/015; A61B 1/05; A61B 1/0676; A61B 17/00234; H04N 23/56; H04N 23/57; A61B 2017/00296; A61B 2017/00371; A61B 2017/22035; A61B 2017/22038; A61M 25/10181; A61M 25/10185; H04N 23/555

(57) ABSTRACT

A portable hand-held medical device includes a body having a gun-type formation that includes a barrel. A guide wire device is housed in the body and is configured to advance and retract through the barrel. The guide wire device carries a bundled arrangement including a video system, an illumination system to support the video system, a fluid irrigation line, and a guide wire mechanism. The bundled arrangement is packaged as a unit in an insertion tube. The tip section of the insertion tube incorporates a camera, a light source, and a fluid irrigation port. A catheter is disposed at the end of the barrel, so that during operation the guide wire device passes through the catheter lumen. The combination guide wire device and catheter enter the urinary tract and perform a bladder evacuation process.

### CROSS-REFERENCE TO RELATED APPLICATIONS

This application is a divisional of U.S. patent application Ser. No. 17/469,621 filed on Sep. 8, 2021, which claims the benefit of U.S. Provisional Patent Application Ser. No. 63/075,345, filed on Sep. 8, 2020, which is incorporated herein in its entirety.

### FIELD OF THE INVENTION

The present invention generally relates to medical instruments and, more particularly, to a device to facilitate the location and extraction of a foreign body from an anatomical cavity or passageway.

### BACKGROUND OF THE INVENTION

Physicians are accustomed to treating illnesses varying in rarity and severity. Most of the less common cases a physician encounters seem bizarre to the general population but are deemed routine work for an experienced physician and other health care workers. For example, broken bones and sprains are not often encountered in one's day-to-day activities—and may not be for many years—but it is frequently treated in Emergency Departments all over. In fact, emergency physicians are the most susceptible to encountering unusual cases, as they are the first department within a hospital to treat the patient experiencing the abnormality.

A common misconception surrounding Emergency Department cases involves a widespread belief that they receive extraordinary and gruesome cases daily, and that emergency department and hospitals everywhere are staffed with every specialist and subspecialis in all fields of medicine ready to assist with every case. Although some hospitals do receive a higher influx of patients that require more immediate assistance, such as hospitals with Level I Trauma Centers, most do not. These hospitals tend to be located in major cities with high population density and in underprivileged neighborhoods where the number of incidents may be higher. However, whether the hospital is in a busy city or a calm rural area or inbetween the two, there are many cases that are present in all kinds of hospitals and must be treated by all.

One seemingly common case that is treated by emergency physicians, but its prevalence remains mostly unnoticed by the rest of the population, are of foreign objects that become stuck in one's body. According to an article published by Beaumont Emergency Center in 2015, entitled “Ten Common ER Visits”, emergency departments all over the world report that one of the most common emergency room visits is due to foreign objects inside the body. As one would logically assume, this may stem from curious children experimenting with items in their vicinity that, by mistake or purposefully, enters their body and become lodged in the child's ear or nose. In adults, food bolus stuck in the esophagus is a common example of foreign body obstruction cases that are mostly dealt with in the emergency department. With a number of cases dealing with adults who used a device, vibrator, or elongated object to elicit a form of gratification.

As such, foreign bodies in a patient's ear, nose, throat, trachea, or rectum are seen among children and adults who present to emergency departments and other health care facilities. To remove a foreign object from a patient in the emergency department, several instruments and procedures can to be used. The problem with the exisiting instruments is that they are not as efficient as they should be because using current instrumentations can lead to failure of removal the foreign body, which is the case in more than 20% of cases, infection, perforation, and other complications that at times can be fatal. For example, a procedure to extract a foreign object from one's nose includes the use of a simple catheter attached to a syringe to get behind the foreign object and pull it out. However, regardless on the location of the foreign object, while attempting to remove the object, the physician may not know of the exact position of the catheter or balloon, and this lead to failure of foreign body removal and increasing the chance of pushing the foreign body deeper, further complicating its removal. This delay in foreign body removal can lead to further complications, such as perforations and infections that at times can be quite serious.

Another example is withdrawal of a foreign body from a patient's esophagus. A common course of treatment is to sedate the patient and blindly pass a foley catheter, hoping it passes the foreign body without lodging it further. This has a suboptimal success rate since the foley catheter and foreign object are not visible to the operator while it is inside the patient's body. When these options do not work, the patient must be admitted to the operating room and have the foreign body endoscopically and/or surgically removed under general anesthesia, exposing the patient to higher risks that involve anesthesia-related side effects.

Accordingly, there is an established need for a medical extraction tool that is able to successfully perform removal of a foreign body, without general anesthesia, and the need of additional specialists in the emergency department such as a general surgeon, ENT, or gastroentrerologist to name few, while minimizing the possibility of complications or trauma to the surrounding passageway, reducing the possibility of further lodging of the foreign body deeper into the anatomical passageway, and accurately navigating the extraction mechanism to ensure its proper positioning relative to the foreign body prior to the withdrawal process. Furthermore, there is a need for such a device that in the hands of physicians and other healthcare personel in an emergency department will be able to successfully remove a foreign body without increasing the patient's health care cost that is typically associated with the inclusion of additional specialists to remove the foreign object, the potential costs associated with transferring a patient to a tertiary care center, and the potential costs associated with booking operating rooms, endoscopy suits, etc., to peform additional procedures to remove the foreign body.

### SUMMARY OF THE INVENTION

The present invention is directed to a portable hand-held device designed to clear a foreign body obstruction from an anatomical passageway. The device includes a catheter reel having a distal portion equipped with a video camera and a light source. The distal portion carries an inflated protective balloon that guards against any interference contact between the patient anatomy and the combination video camera and light source as an added measure to prevent incidental trauma to the surrounding tissue or membrane surrounding the foreign object, during the deployment phase when the catheter navigates through the passageway to reach the obstruction site.

During the extraction procedure, the catheter is inserted into the obstructed passageway and advanced through it until the distal portion encounters the foreign body obstruction. The distal portion of the catheter is steered and otherwise navigated to the rear or posterior side of the foreign body. At this point, an extraction balloon, which until now is deflated and carried by the distal portion, is inflated. Subsequent retraction of the catheter induces the foreign body to move in an egress direction in response to the pushing exertion that the inflated extraction balloon and the retracting device imposes on the foreign body. In one form, the device is configured as a gun-type formation with selector controls to manage the operation of the catheter and balloons. The combination video camera and light source enable the operator to visually view the progress of the catheter advancement and positioning, particularly to recognize the encounter with the foreign body and to help guide the subsequent steering of the distal portion of the catheter to the far or posterior side of the foreign body before inflation of the extraction balloon.

Introducing a first embodiment of the invention, the present invention consists of medical device for extracting a foreign body from an anatomical passageway, comprising:
a housing including a handle portion having at least two triggers;
a deployable catheter comprising an elongated extendable body, a camera, and a light source, the camera and the light source disposable about a distal end of the deployable catheter; and
at least one balloon disposable about the distal end of the deployable catheter, inferior to the camera and the light source.

In another aspect, at least one trigger can control the inflation and deflation of the inflatable balloon.

In another aspect, at least one trigger can control the position of the distal end of the deployable catheter.

In another aspect, the camera and light source are electrically connected to a display device.

In another aspect, the medical device may comprise an irrigation lumen controllable by at least one trigger.

In another aspect, the balloon may be a protective, non-deflateable, balloon.

In another aspect, the balloon may be an inflatable balloon.

In yet another aspect, the catheter may include a guide wire controllable by the at least one trigger.

In another aspect, the distal end of the catheter is non-rigid.

In a second embodiment the present invention consists of a method of extracting a foreign body from an anatomical passageway, comprising the steps of:
providing a catheter having a distal portion carrying an inflated guard balloon, a light source, a video camera, and a deflated extraction balloon;
inserting the catheter into the anatomical passageway;
navigating the catheter through the passageway until the distal portion encounters the foreign body, using a video feed supplied by the video camera to accompany the navigation operation;
steering the catheter relative to the foreign body until at least the distal portion of the catheter is positioned behind the foreign body at a posterior side thereof;
inflating the extraction balloon carried by the distal portion of the catheter; and
retracting the catheter.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a front, upper perspective showing a first embodiment of the medical extraction device of the present invention;

FIG. 2 presents a front, upper perspective showing the first embodiment of the medical extraction device of the present invention and further illustrating in magnified view the distal tip portion of the catheter portion of the device;

FIG. 3 presents a front, upper perspective showing the first embodiment of the medical extraction device of the present invention, further illustrating the catheter portion in an advancing mode of operation and the mechanism to control the catheter movement;

FIG. 4 presents a side elevation section view of an illustrative anatomical passageway with a foreign body obstruction, illustrating the operation of the medical extraction device of the present invention and specifically depicting the approach of the catheter portion towards the foreign body as enhanced by a light illuminator and a live feed video stream camera;

FIGS. 4A-C present a series of views of the obstructed anatomical passageway originally disclosed in FIG. 4 to illustrate the process performed by the medical extraction device of the present invention to remove the foreign body, illustrating in sequence the navigation of the distal portion of the catheter past and behind the foreign body (FIG. 4A), inflation of a balloon on the far side of the foreign body to facilitate subsequent dislodging and pushing of the foreign body back out the passageway (FIG. 4B), and extraction of the foreign body by retraction of the catheter to induce movement of the foreign body in response to the urging of the withdrawn balloon (FIG. 4C);

FIG. 5 is a diagrammatic view of a treatment system employing the first embodiment of the medical extraction device of the present invention, illustrating integration of the medical extraction device with a video monitor display to allow the operator to view the operation of the catheter portion in real time as part of the foreign body removal procedure; and

FIG. 6 is a block diagram flowchart of one illustrative process employing the present invention to extract a foreign body obstruction.

Like reference numerals refer to like parts throughout the several views of the drawings.

### DETAILED DESCRIPTION

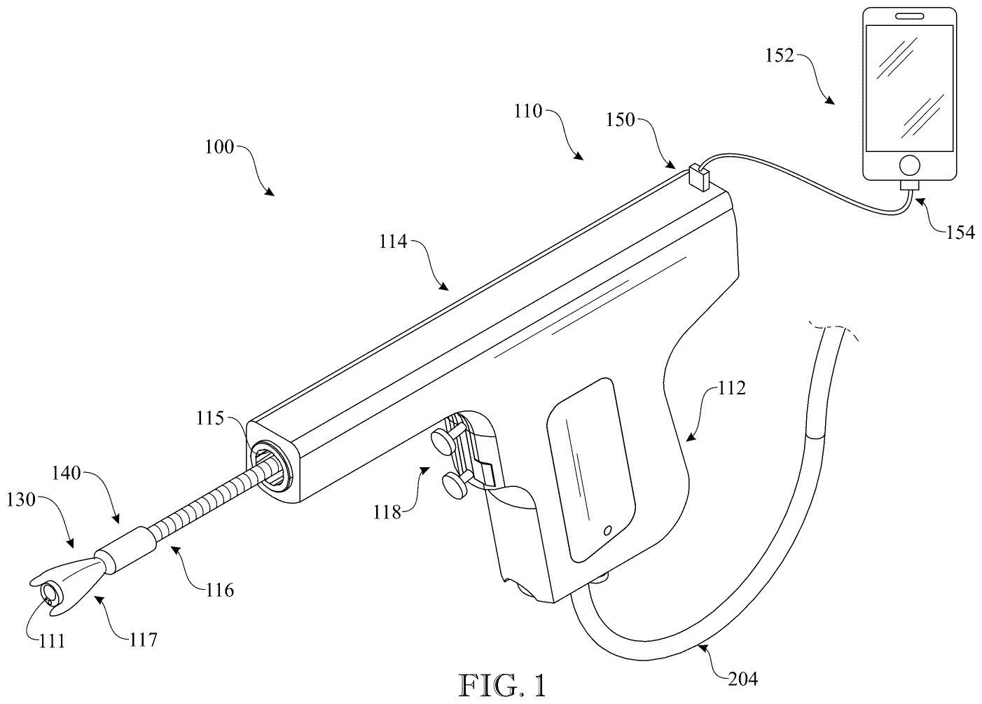

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a method and device to perform an extraction procedure that locates and removes a foreign body obstruction located in an anatomical passageway.

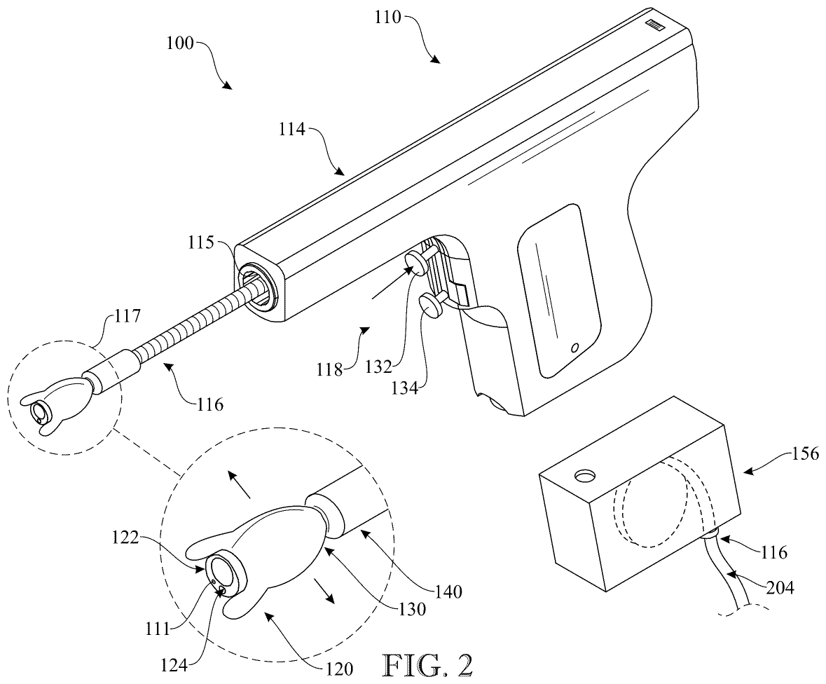

Referring initially to FIGS. 1 and 2, a hand-held, portable medical device is generally illustrated at 100, according to a first embodiment of the present invention. As discussed further, the medical device 100 facilitates the removal of a foreign body obstruction located in a patient's anatomical cavity or passageway, such as, but not limited to, the ear, nose, esophagus, trachea, vagina, or rectum.

The medical device 100 includes a tool or instrument body generally illustrated at 110. In one implementation, the instrument body 110 is preferably configured in the form of a gun-type configuration that includes, in combination, a grip handle 112, a barrel portion 114 terminating in a muzzle-type opening 115, a catheter portion 116 loaded into barrel portion 114 and which selectively advances/retracts through opening 115, a distal tip portion 117 defined at the terminus free end of catheter portion 116, and a control mechanism generally illustrated at 118 that directs and otherwise manages the movement of catheter portion 116, including distal tip portion 117. The medical device 100, in various forms, can be considered a tool, instrument, or implement. The catheter portion 116 is preferably provided in the form of a reel loaded into instrument body 110, which can be selectively extended (advanced) and retracted.

In an alternative embodiment, the medical device may include an internal or external housing 156 that houses a predetermined loaded length of the catheter 116. In this exemplary form, the size, shape, diameter, and pliability of the catheter will depend on whether the device is being used to dislodge a foreign object, for example, from a nose, rectum, vagina, or an esophagus. Put differently, the length, diameter, shape, and pliability of the catheter housed inside of the catheter housing will depend on the anatomical location of the foreign object. In yet another embodiment, the device 100 may comprise an instrument body 110 in the form of a gun-type configuration that includes, in combination, a grip handle 112, a barrel portion 114 terminating in a muzzle-type opening 115, a selectively attachable catheter portion 116, and a control mechanism 118 that directs and otherwise manages the movement of catheter portion 116, including distal tip portion 117. Provided the circumstance the device 100 may be utilized in, for instance inside of a rectum, esophagus, nasal cavity, or the like, the device may be either completely disposable (i.e., the entire device is thrown away after it is used), or partially disposable (i.e., the catheter or any other portion of the device that makes contact with the internal anatomy of the patient is thrown away after it is used).

The catheter portion 116, in one form, has a tubular construction including a conduit lumen defining an interior space that spans the length of catheter portion 116. In some embodiments, the catheter portion may include an irrigation lumen 111 (see FIG. 1) to clear tissue debris, blood, and or tissue to facilitate the procedure. The catheter portion 116 can be made of any suitable material, but particularly one that is biocompatible in view of the human application for medical device 100.

The construction of catheter portion 116 can be tailored to the particular application (i.e., the anatomical location of the foreign body). During operation, catheter portion 116, with distal portion 117 as the leading element, is introduced into the passageway or cavity of a patient where a suspected foreign body is located, and then advanced (navigated) to reach the obstruction site. During this navigation process, the catheter portion 117 is unwound and extended from its housed location in instrument body 110. In certain applications, such as those involving shorter passageways (ear, nose) requiring less maneuvering of distal portion 117, the catheter portion 116 may have a rigid or semi-rigid quality. In other applications, such as those involving longer passageways (esophagus, trachea) and/or a non-linear access route to the foreign body obstruction, the catheter portion 116 may have a more flexible, length, diameter, and other related construction properties to accommodate the curvilinear or serpentine path that catheter portion 116 must navigate to reach the foreign body obstruction site.

The medical device 100 includes a viewing and navigation unit generally illustrated at 120. The viewing unit 120 includes a set of features incorporated into catheter portion 116 (at distal portion 117), which enable the operator to view the interior environment of the anatomical passageway during the navigation and travel of catheter portion 116 to the foreign body obstruction site. According to the present invention, the viewing unit 120 of medical device 100 includes, in combination, a video camera 122 and a light source 124, cooperatively housed at the distal portion 117 of catheter portion 116. In one form, the video camera 122 and light source 124 are disposed at the leading edge of distal portion 117, although any other suitable location is possible. The light source 124 can be configured in any suitable conventional form, for example as an LED. The light source 124 provides illumination of the anatomical passageway 302 once catheter portion 116 is admitted into the patient's anatomy, functioning as a type of headlight. The light source 124 can be powered by a battery located in instrument body 110, using a connection that traverses the conduit lumen of catheter portion 116. Although light source 124 is especially helpful to provide forward illumination of passageway 302 to assist the operator in guiding the advancing progress of catheter portion 116 and locating foreign body 300, the ability of distal portion 117 to be steered and manipulated into a variety of directions means that light source 124 can effectively provide omni-directional illumination, not just pointing straight ahead. The video camera 122 provides a real-time video feed capturing video images of the area where distal portion 117 is pointing, particularly the space in front of catheter portion 116. The video camera 122 can be configured in any suitable conventional form well known to those skilled in the art.

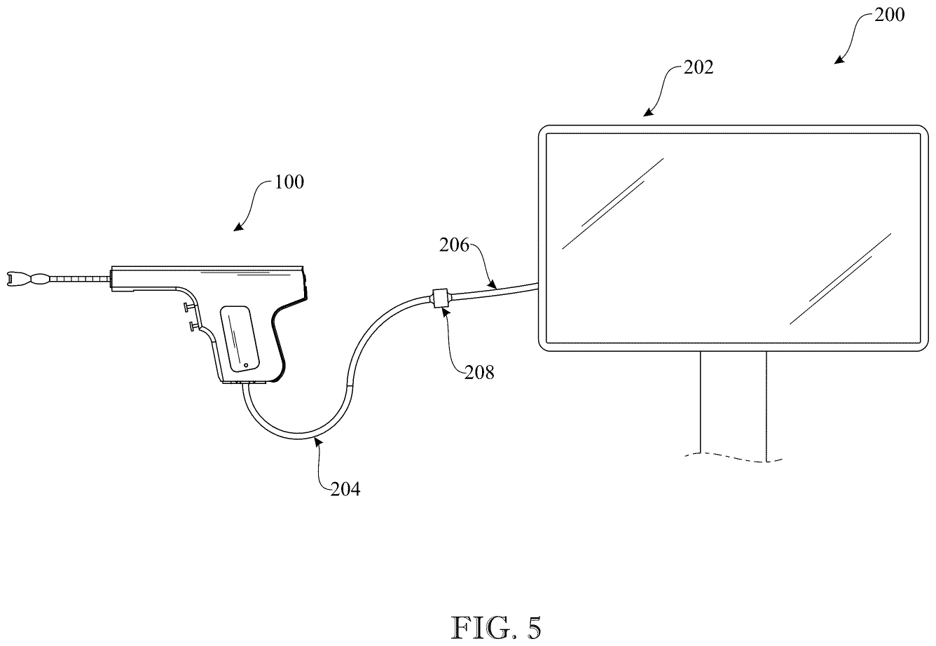

Referring briefly to FIG. 5, the medical device 100 is integrated with a video display system 200. The video display system 200 receives and displays the video images acquired by video camera 122 integrated into medical device 100. For this purpose, an appropriate communication line is provided between video camera 122 and a display monitor or screen 202 of video display system 200. For example, in one implementation, a fiber optic line or cable 204 extends from video camera 122, passes through the lumen of catheter portion 116, and extends out of instrumentation body 110. The fiber optic cable 204 is connected to a video input cable 206 of display monitor 202 using a suitable adapter or coupler 208. In another exemplary embodiment, yet harmonious with the present invention, the medical device may include an electrical female video component 150 disposed about a top portion of the instrument body 110 that is used to connect a portable electronic device 152 to the medical device, via a male electrical connector 154. The portable electronic device may include, but is not limited to a smart phone, a tablet, a laptop, a portable video monitor, or the like. In this particular embodiment, the medical device is completely portable. The optic line extending from video camera 122 passes through the lumen of catheter portion 116 and connects directly to the electrical female video component to transmit live images to the portable video monitor connected thereto. In an alternative embodiment, the video camera 122 includes the necessary components, known in the art, to wirelessly broadcast a video signal that is picked up by the electronic device 152. The signal may be transmitted over Wi-Fi, Bluetooth, or any other known frequency or transmission technique.

Referring again to FIG. 2, the distal portion 117 of catheter portion 116 houses and otherwise carries a protective balloon 130 that functions as a bumper or guard component designed to protect the surrounding anatomical structures (e.g., tympanic membrane) from contact with the viewing unit 120 (video camera 122 and light source 124) as catheter portion 116 passes through an anatomical passageway. As shown, the protective balloon 130 includes a concave shape, resembling an umbrella, with the outer edges extending beyond the viewing unit. In particular, the distal portion 117 carries an inflated protective balloon 130 that guards against any interference contact between the patient anatomy and the combination video camera 122 and light source 124, during intervention and treatment.

For this purpose, protective balloon 130, upon inflation, has a size and shape defining at least one cross-sectional planar surface that is larger than the cross-sectional arca occupied by viewing unit 120. In this manner, as catheter portion 116 passes through an anatomical passageway, any close approach between catheter portion 116 (distal portion 117) and the surrounding tissue will create a contact between the tissue and protective balloon 130, maintaining the viewing unit 120 a safe distance away. In one form, protective balloon 130 is located immediately behind viewing unit 120. The protective balloon 130 is preferably maintained in an inflated state throughout the operation of medical device 100, but at least during the intervention and treatment period when catheter portion 116 is in the patient's body. The protective balloon 130 can be provided in an inflated condition in its off-the-shelf configuration distally to an extraction balloon 140. For this purpose, the instrument body 110 can house a valve-controlled cartridge of pressurized air, which is connected by a suitable pneumatic line to the originally deflated extraction balloon 140. At the appropriate time, the operator initiates inflation of extraction balloon 140 by activation of the air valve using a control selector such as a push-button trigger 132 configured in control mechanism 118. Any suitable means known to those skilled in the art can be used to implement the inflation feature of extraction balloon 140. The extraction and protective balloon 140, 130 can be made of any suitable material and constructed in a variety of appropriate sizes and shapes suitable to the medical application. One inflated shape, for example, includes an oval-like geometry; however, alternative shapes may be employed without departing from the scope of the invention. For instance, depending on the location of the foreign body and the foreign body itself the shape of the balloon may vary to facilitate the removal of the foreign body.

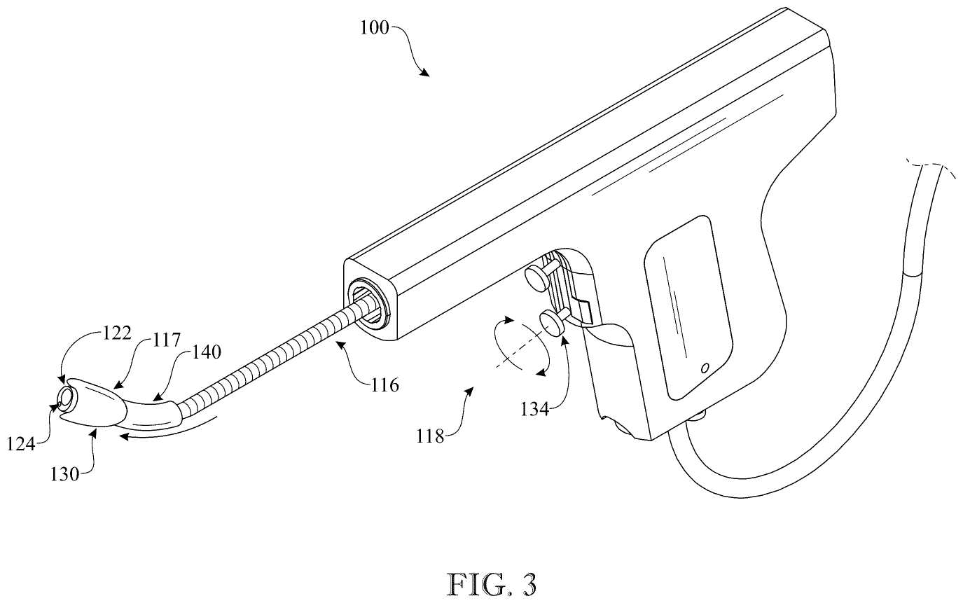

Referring now to FIG. 3, with continuing reference to FIGS. 1 and 2, medical device 100 further includes a maneuvering feature that enables the operator to selectively and controllably manipulate both the longitudinal movement of catheter portion 116 (i.c., advance and retract) and the movement of distal portion 117 through the plane orthogonal to the long axis of catheter portion 116 (c.g., lateral and rotational movement). Generally, this maneuvering feature supports the capability to drive catheter portion 116 in its axial movement and to drive distal portion 117 in its omni-directional movement. In this manner, the catheter portion 116 can be moved and otherwise guided through the patient's anatomy until it encounters the foreign body obstruction, and then the distal portion 117 can be steered into a position suitable for performing the extraction process, as discussed further. The medical device 100 includes a suitable mechanism to control the movement of distal portion 117. The medical device 100 includes a control feature in control mechanism 118, such as an exemplary actuator-type selector 134, to direct the operation of the mechanism that maneuvers distal portion 117. In one form, the actuator-type selector 134 includes a trigger- like functionality (in/out toggle positions) to selectively control the advancement and retraction of catheter portion 116 according to the selected toggle position, and further includes a dial-like functionality (rotate the dial knob of selector 134) to selectively control the maneuvered movement of distal portion 117. For example, rotation of the dial knob of selector 134 produces a corresponding and proportional rotation of distal portion 117. The distal portion 117 will feature the ability to be bendable in all directions. In yet another embodiment, the control mechanism 118 of the device 100 may include a third selector (not shown) that controls the irrigation lumen 111 on catheter 116.

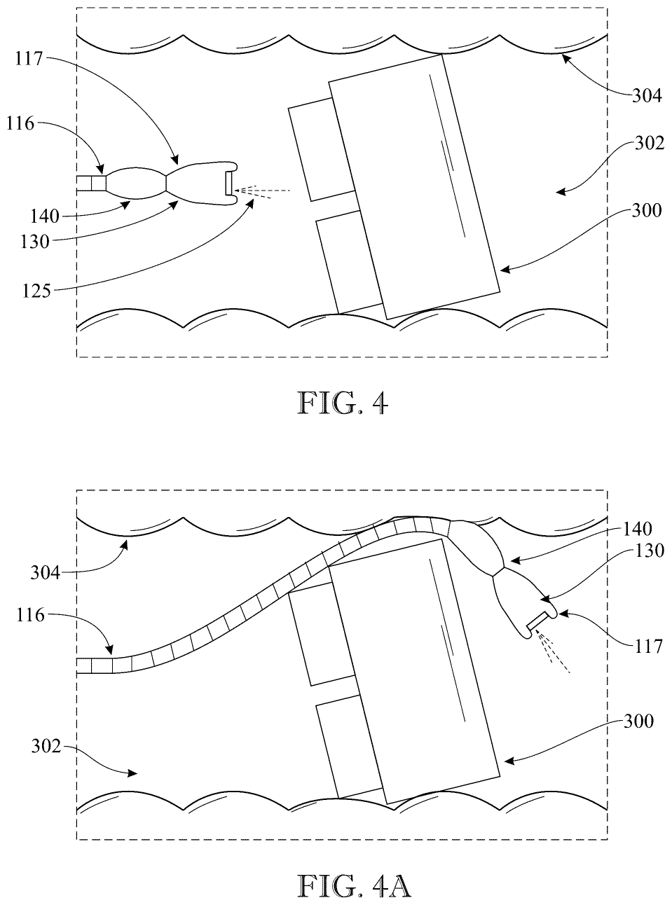

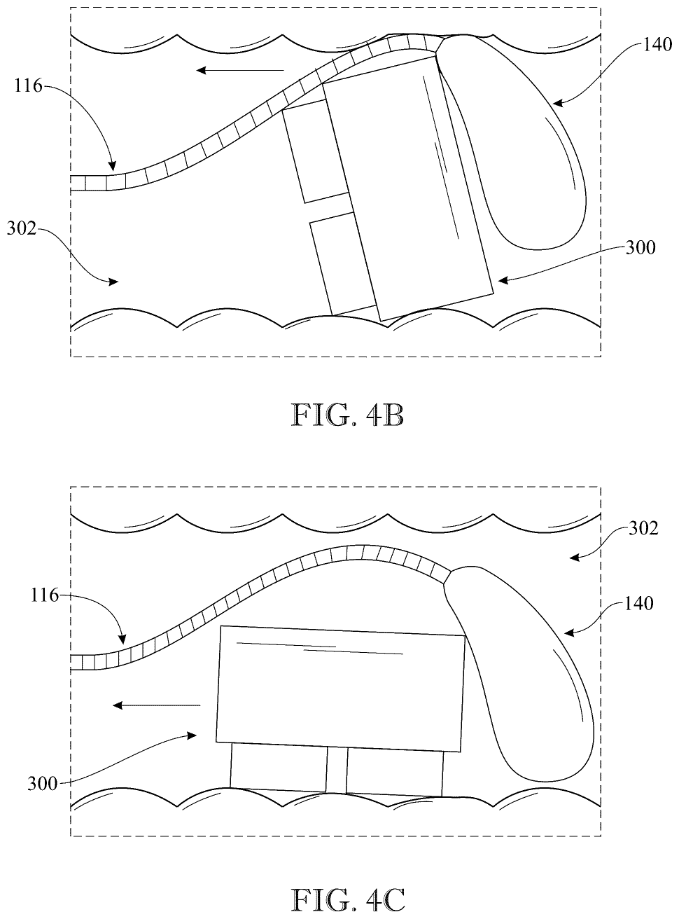

Referring now to FIGS. 4 and 4A-C, an exemplary operation of medical device 100 is described. The medical device 100 is deployed to help locate, inspect, remove, and otherwise extract a foreign body 300 causing an obstruction in an anatomical passageway 302 lined with illustrative tissue or cartilage 304 (FIG. 4). The foreign body 300 can represent a variety of articles or objects, such as beads, beans, cotton, insects, paper, coin, metal, marble, food particles, and toys. Prior to deployment of medical device 100, the viewing unit 120 is activated, which turns on video camera 122 and light source 124 (or connects and turns on the portable video monitor 154 connectable to the medical device, as shown in FIG. 1). When activated, light source 124 produces an illustrative illumination pattern 125. Additionally, the protective balloon 130 is inflated, if not otherwise provided in this condition. To initiate the intervention, the distal portion 117 of catheter portion 116 is introduced into passageway 302, specifically by entering the relevant bodily orifice (nostril, car canal, esophagus, vagina, anus) that offers access to the obstructed passageway 302. The user then controls the advancement of catheter portion 116 through passageway 302 via activation of the appropriate selector (e.g., catheter portion 116 is unwound from its reeled storage condition in instrument body 110). The speed of catheter advancement can be controlled by the user.

At the same time as catheter portion 116 is unrecled and extended further into passageway 302, the user is simultaneously observing the video images displayed on monitor 202 (or electronic device 152, depending on which display type is being used), which represent the live video feed acquired by video camera 122. The user is monitoring the video feed to locate the presence of foreign body 300, whose specific location is otherwise unknown apart from its presence in a particular anatomical organ (c.g., nose, car, throat). The video feed also helps the user to facilitate a safe and trauma-free guided navigation of catheter portion 116 through passageway 302 until it encounters foreign body 300, a task especially useful when steering through a serpentine bodily pathway. If necessary, the user may use the irrigation lumen to clear debri, tissue, or other particles obstructing the live video feed.

As shown in FIG. 4, after a suitable advancement of catheter portion 116 in passageway 302, the distal portion 117 approaches and confronts foreign body 300 at its anterior or near side, which comes into view as light source 124 illuminates foreign body 300. At this point, the user conducts an assessment of the partial or total obstruction created by foreign body 300, examining factors such as the type, positioning, orientation, and geometry (size, shape) of foreign body 300. This assessment is used to determine the best course of action to extract foreign body 300. In the illustrative depiction of FIG. 4, foreign body 300 is tilted forward and spans diametrically across passageway 302. The orientation of foreign body 300 suggests a strategy for removal that entails pushing the foreign body 300 from behind (i.c., an exertion directed from the posterior side to the anterior side of foreign body 300), in order to induce the foreign body 300 to fall further in the direction of its tilt until it comes to rest in a stable, balanced orientation more conducive to removal. It should be apparent that the extraction process described in connection with FIG. 4 is merely illustrative and should not be considered in limitation of the present invention, as the removal process can be modified and otherwise adapted to the particular obstruction under examination.

Referring now to FIGS. 4A-C, the strategy adopted by the user to extract foreign body 300 involves the need to position distal portion 117 of catheter portion 116 at the posterior or far side of foreign body 300. As discussed further, due to the forward tilting orientation of foreign body 300, the assisted fall or toppling of foreign body 300 is more readily accomplished by pushing it from behind at a point creating maximum torque moment, which in this case is at the upper end. Accordingly, the distal portion 117 of catheter portion 116 is appropriately steered so that it travels between tissue 304 and the upper end of foreign body 300, eventually emerging on the posterior or far side of foreign body 300 (FIG. 4A). During this steering process, the inflated protective balloon 130 will contact and slide along the exposed surface of tissue 304, preventing the combination of video camera 122 and light source 124 from engaging the tissue lining 304 and potentially creating a trauma (c.g., perforation or tearing). As shown, for the illustrative foreign body obstruction, the gap between tissue lining 304 and foreign body 300 progressively narrows in the front-to-back (anterior-to-posterior) direction at the upper end of foreign body 300, in its original, lodged position within passageway 302. However, due to the elastic quality of tissuc lining 304 and protective balloon 130, the protective balloon 130 is able to flex and sufficiently displace tissue lining 304 in the radial direction as it travels axially above foreign body 300, so that the distal portion 117 of catheter portion 116 can successfully navigate through the increasingly narrow clearance space.

Eventually, once protective balloon 130 clears the foreign body 300 below, the distal portion 117 enters the posterior space of foreign body 300 (FIG. 4A). Throughout this steering process calculated to position distal portion 117 at the posterior side of foreign body 300, the user continues to visually monitor the progress of catheter portion 116, via video display system 200, to ensure that distal portion 117 is guided in the proper direction relative to foreign body 300. It should be apparent that the guiding and steering mechanism (i.c., the joy stick or knob 134) used to manipulate and otherwise maneuver distal portion 117 can execute any type of snaking travel required to move and reposition distal portion 117 of catheter portion 116 behind foreign body 300.

Referring more particularly to FIGS. 4B-C, medical device 100 incorporates an inflatable extraction balloon or sac 140, according to another feature of the present invention. The extraction balloon 140 is carried by the distal portion 117 of catheter portion 116 in a deflated state. Once distal portion 117 of catheter portion 116 is positioned behind foreign body 300, the extraction balloon 140 is inflated (FIG. 4B). For this purpose, the instrument body 110 of medical device 100 is equipped with a controllable, valve-operated cartridge or dispenser of pressurized air that is pneumatically connected to extraction balloon 140. The operator can actuate this inflation of extraction balloon 140 using selector 132 (FIG. 2), for example, when control mechanism 118 is configured for this purpose by fashioning selector 132 as the control feature for regulating inflation of extraction balloon 140. The pneumatic control of extraction balloon 140 can employ any conventional means known to those skilled in the art.

The present invention deploys an extraction strategy for withdrawing foreign body 300 from passageway 302 that uses a principle of action involving the assisted displacement of foreign body 300, which is enacted by pushing it from behind with extraction balloon 140. In order to reposition foreign body 300 into an orientation more conducive to push-assisted travel, it is preferable to first topple foreign body 300 from its lodged, tilted position. For this purpose, once distal portion 117 is maneuvered into place behind foreign body 300 and the extraction balloon 140 is inflated (FIG. 4B), the catheter portion 116 is retracted an amount adequate to create a pushing action on foreign body 300 that is sufficient to topple, shift, or otherwise move the foreign body 300 and reorient it in a more stable position suitable for removal (FIG. 4C). It should be apparent that this preliminary toppling step will not always be needed. Rather, the depiction of how foreign body 300 is lodged in passageway 302 in a slanted fashion is merely illustrative; it will not always be necessary to first dislodge foreign body 300 from its original placement before being withdrawn, although this scenario can frequently occur depending on the configuration of the obstruction created by foreign body 300. It should be understood that the use of extraction balloon 140 to reposition foreign body 300 prior to further displacement and removal (i.c., the transition from FIG. 4B to 4C) is more broadly representative of any suitable technique to reposition foreign body 300 prior to withdrawal, which can be performed by the push-type exertion of extraction balloon 140 against foreign body 300 from behind.

With continued reference to FIGS. 4B-C, the extraction process now proceeds with withdrawal of foreign body 300 from passageway 302 by driving or urging foreign body 300 to displace in the longitudinal direction of passageway 302. First, however, catheter portion 116 is maneuvered as needed to ensure that the inflated extraction balloon 140 is adequately positioned at the posterior side behind the repositioned foreign body 300 (FIG. 4C). The operator can now commence the withdrawal or extraction of foreign body 300 by appropriately retracting catheter portion 116 or pulling on the catheter portion and medical insturment. The effect of this retraction of catheter portion 116 is to simultaneously retract the distal portion 117 carrying the inflated extraction balloon 140, which, because of the intimate engagement between extraction balloon 140 and foreign body 300 at the rear or posterior side of foreign body 300, induces a forward sliding motion in foreign body 300 in tandem with the movement of extraction balloon 140.

In particular, the inflated extraction balloon 140, as it is drawn forward by the pulling action of the retracting catheter portion 116, exerts a pushing action on foreign body 300 that propels and otherwise urges the foreign body 300 to slidingly displace in the longitudinal or egress direction of passageway 302, towards the bodily orifice where the foreign body 300 was originally admitted. As the catheter portion 116 continues to be retracted, the inflated extraction balloon 140 maintains its driving, propelling, pushing exertion against foreign body 300. In particular, subsequent and continued retraction of the catheter portion 116 induces the foreign body 300 to move in an egress direction in response to the pushing exertion that the inflated extraction balloon 140 imposes on the foreign body 300. The foreign body 300, in response to this continuous propelling action exerted by the moving extraction balloon 140, is eventually fully withdrawn from passageway 302 until it reaches the relevant body orifice, where it can be removed from the patient to complete the extraction process.

The inflatable extraction balloon 140 can be adapted, in terms of size, shape, and geometry, to accommodate the type of foreign body 300 intended for extraction. For example, a more elongate size is fitting for the configuration of foreign body 300 illustratively depicted in FIG. 4, in order to ensure that the inflated extraction balloon 140 can adequately drape over the rear end of foreign body 300 to promote the success of the pushing action exerted by balloon 140 against foreign body 300 once catheter portion 116 undergoes retraction. Any suitable structure and material construction can be used to implement balloon 140, where the form preferably features a flexibility and elasticity and is capable of holding pressurized air in an inflated state.

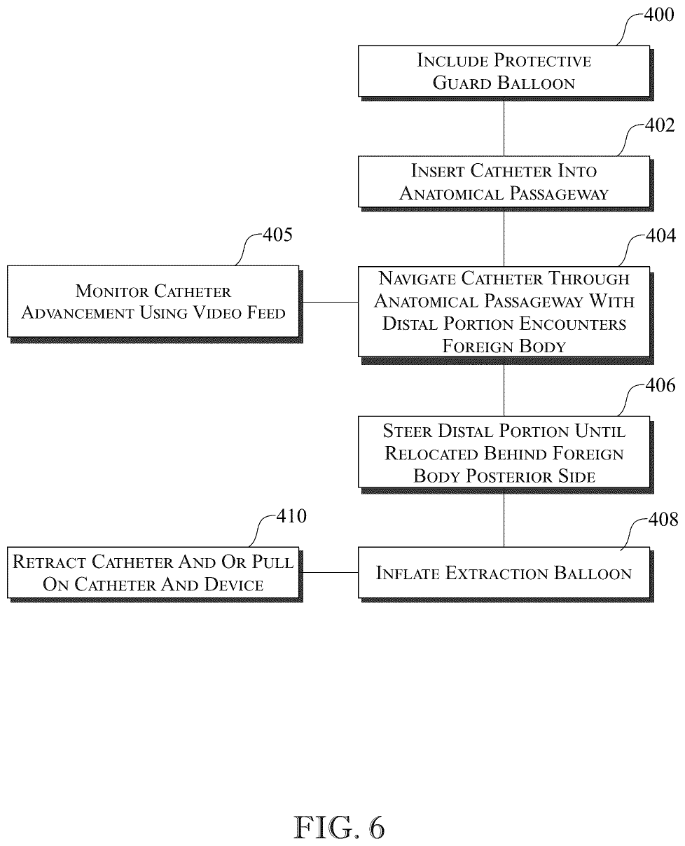

Referring to FIG. 6, a flowchart is disclosed to describe the features of the procedure that, upon execution, deploys medical device 100 in conjunction with video display system 200 to perform an extraction procedure on foreign body 300 lodged in anatomical passageway 302. In a preliminary set-up task, prior to introduction of catheter portion 116 into the patient, the protective guard balloon 130 is inflated, if needed (step 400). However, in a preferred embodiment the protective balloon 130 is provided in a pre-inflated state. The catheter portion 116 is then inserted into the passageway 302 (step 402). Following insertion, the catheter portion 116 is guided and otherwise navigated to the foreign body obstruction site, using the real-time video images of passageway 302 obtained from video camera 122 as feedback to facilitate the guiding process (steps 404, 405). Once the foreign body 300 comes into view, accompanied by the distal portion 117 of catheter portion 116 encountering foreign body 300 at its anterior side, the catheter portion 116 is steered around foreign body 300 until at least distal portion 117 is relocated behind or at the posterior side of foreign body 300 (step 406). The extraction balloon 140 is then inflated (step 408). The catheter portion 116 is then retracted or pulled (under user control) to induce withdrawal of foreign body 300 along passageway 302, in response to a pushing action exerted by the inflated extracted balloon 140 against foreign body 300 as the extraction balloon 140 moves in tandem with the retracting catheter portion 116 and distal portion 117 (step 410).

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A method of using medical device to extract a foreign body from an anatomical passageway, comprising the steps of:
providing a medical device comprising,
a housing including a handle portion having at least two triggers;
a deployable catheter comprising an elongated extendable body and a viewing unit, the viewing unit disposable about a distal end of the deployable catheter; and
a least one balloon disposable about the distal end of the deployable catheter, inferior positioned to the viewing unit;
inserting the catheter into an anatomical passageway,
wherein the distal end of the catheter is positioned at a posterior side of the foreign body;
inflating the balloon to apply a force against the foreign body to move the foreign body from its current state; and
retracting the catheter from the anatomical passageway to remove the foreign body.

2. The method of claim 1, wherein at least one trigger controls the inflation and deflation of the inflatable balloon.

3. The method of claim 2, wherein the trigger communicates to a pneumatic line that connects to a cartridge of pressurized gas housed inside the housing, the cartridge having a control valve connected to head for controlling release of the pressurized gas, and wherein depressing the trigger engages the control valve to release pressurized gas and inflate the inflatable balloon.

4. The method of claim 1, wherein the viewing unit includes a camera, and a light source.

5. The method of claim 1, wherein at least one trigger controls the deployment of the catheter and positional movement of the distal end of the catheter.

6. The method of claim 4, wherein the camera is connectable to an electronic device through a connection port located on an upper surface of the housing of the medical device.

7. The method of claim 1, wherein the medical device further comprises an irrigation lumen.

8. The method of claim 7, wherein the irrigation lumen is controlled by at least one trigger.

9. The method of claim 1, wherein the deployable catheter includes a guide wire controllable by at least one trigger.

10. The method of claim 4, wherein the camera is wirelessly connected to an electronic device, the camera capable of transmitting real-time video transmissions to a viewing screen on the electronic device.

11. The method of claim 1, wherein the at least one balloon is inflatable to a size and shape including at least one cross-sectional planar surface that is larger than the cross-sectional area occupied by the viewing unit.

12. The method of claim 1, wherein the handle portion of the medical device includes an internal housing portion for storing a pre-determined length amount of the deployable catheter.

13. The method of claim 1, further comprising an external housing attachable to the handle portion of the medical device, wherein the external housing stores a pre-determined length amount of the deployable catheter.

14. The method of claim 1, wherein the medical device is entirely disposable after its usage to remove the foreign body from the anatomical passageway.

15. The method of claim 1, wherein the deployable catheter is disposable after its usage to remove the foreign body from the anatomical passageway.

16. The method of claim 1, wherein the balloon includes an umbrella-shaped body having an outer edge that extends beyond the viewing unit.
